(19) United States
(12) Patent Application Publication
(10) Pub. No.: US 2023/0293560 A1
(43) Pub. Date: Sep. 21, 2023

(54) METHOD FOR MODULATING THE BLADDER MICROBIOME TO IMPROVE BLADDER HEALTH

(71) Applicant: Kimberly-Clark Worldwide, Inc., Neenah, WI (US)

(72) Inventors: Kathleen C. Engelbrecht, Kaukauna, WI (US); Elizaabeth M. Doney, Appleton, WI (US); David W. Koenig, Menasha, WI (US); Stephen Quirk, Alpharetta, GA (US); Alexnder J. Horkman, Neenah, WI (US); Jane Ballesteros, Oshkosh, WI (US)

(21) Appl. No.: 18/017,297

(22) PCT Filed: Jul. 23, 2021
(86) PCT No.: PCT/US21/42890

§ 371 Date: Jan. 20, 2023

Related U.S. Application Data
(60) Provisional application No. 63/055,369, filed on Jul. 23, 2020.

(51) Int. Cl. A61K 31/7016; A61P 13/10

(52) U.S. Cl. CPC A61K 31/7016; A61P 13/10

(57) ABSTRACT

Methods and compositions for modulating a bladder microbiome in a subject to improve bladder health are disclosed. The method can include providing a composition including a carrier and a bladder therapeutic agent. The bladder therapeutic agent can include isomaltulose, dextrin type 1, dextrin type 2, or combinations thereof. The method can further include administering the composition to a urogenital region of the subject. The method can include promoting a growth of Lactobacillus crispatus relative to Streptococcus anginosus within the bladder microbiome to modulate the bladder microbiome to improve bladder health.

### BACKGROUND OF THE DISCLOSURE

Globally, about 800 million people have urinary incontinence (UI) and 70% are female. Urgency urinary incontinence (UUI) is one form of UI. Overactive bladder (OAB) can include muscle spasms of bladder muscles that can make a person feel as though they need to urinate, but may not leak urine. In some patients having UUI or OAB may experience the sensation of the need to instantly urinate regardless of whether the bladder is full. The sensing of a bladder being filled can involve various parts of a person's nervous system, and can eventually lead to contraction of bladder muscles, specifically the detrusor muscle, during micturition.

Despite the large number of people with UI, there is a lack of sufficient long term treatment. Various products exist to provide the ability to possibly lessen or manage incontinence symptoms without medical intervention, however, these products may involve insertion of various physical products or providing various stimulations near a person's bladder.

Additionally, urinary tract infections (UTI) can be rather common infections, especially within women. UTIs most commonly infect a women's bladder and/or urethra and in some instances, can be painful and cause other negative symptoms.

Contrary to dogma, it has been established that the bladder contains bacteria and that the presence of known healthy bacteria (Lactobacillus sp.) in the bladder may be associated with a lack of urge incontinence and overactive bladder symptoms. If the healthy bacteria could be supported, then the symptoms of urge incontinence and overactive bladder may be reduced, or possibly prevented. However, bacteria species in one microbiome of a subject do not necessarily respond to therapeutic agents in the same fashion as bacteria species from another microbiome of the subject.

Accordingly, a need exists for compositions and methods for modulating the bladder microbiome to improve bladder health in a subject. A need also exists for compositions and methods for preventing or treating incontinence, overactive bladder, or urinary tract infections in a subject.

### SUMMARY OF THE DISCLOSURE

It has now been surprisingly discovered that the bladder microbiome of a subject may be modulated to help the growth of certain commensal bacteria, such as Lactobacilli, yet maintaining or hindering the growth of certain pathogenic bacteria, such as Streptococcus, with certain bladder therapeutic agents to improve bladder health. As a result, it has been discovered that certain bladder therapeutic agents can be administered to a subject to treat or prevent certain conditions of the bladder, such as UUI, OAB, and UTI.

In one aspect, a method for modulating a bladder microbiome in a subject to improve bladder health is provided. The method can include providing a composition. The composition can include a carrier and a bladder therapeutic agent. The bladder therapeutic agent can include isomaltulose. The method can further include administering the composition to a urogenital region of the subject. The method can additionally include promoting a growth of Lactobacillus crispatus relative to Streptococcus anginosus within the bladder microbiome to modulate the bladder microbiome to improve bladder health.

In another aspect, a method for preventing or treating overactive bladder, urinary urge incontinence, or urinary tract infections in a subject is provided. The method can include providing a composition. The composition can include a carrier and a bladder therapeutic agent. The bladder therapeutic agent can include isomaltulose. The method can further include administering the composition to a urogenital region of the subject to prevent or treat overactive bladder, urinary urge incontinence, or urinary tract infections in the subject.

### Definitions

As used herein, the term “inhibit” generally means to reduce by a measurable amount or to prevent entirely.

As used herein, the term “urogenital” refers to the vulva, vagina, urinary tract, bladder, and surrounding areas.

As used herein, the terms “effective amount” and “therapeutic amount” is an amount sufficient to inactivate, but not necessarily kill, pathogenic microorganisms that may be responsible for or lead to infection of the bladder. In fact, although not required, it may be desired to use a concentration that does not significantly affect or inhibit the growth characteristics of the normal bladder flora or otherwise significantly irritate the bladder tissue when used at inhibitory, noncytotoxic, or clinical concentrations. For example, a bladder therapeutic agent can be desirably employed at a concentration of about 0.01 to about 7.5 wt/vol %, in some embodiments from about 0.1 to about 5.0 wt/vol %, in some embodiments from about 0.2 to about 2.0 wt/vol %, and in some embodiments from about 0.5 to about 1.5 wt/vol %. It should be understood that the dosage may vary with the age, condition, and type of infection suffered by the patient, and may be readily determined by one of skill in the art.

As used herein, the term “therapeutic effect” refers to the ability of the compositions and formulations of the present disclosure to stimulate the growth of L. crispatus relative S. anginosus measured according to the therapeutic effect protocol described below. The therapeutic effect can be expressed as a ratio of L. crispatus to S. anginosus and is desirably greater than about 100, more preferably greater than about 500 and more desirably greater than about 1,000.

As used herein, the designation “wt/vol %” or “wt/vol” refers to the value obtained by dividing the weight of a substance (in grams) by the volume of the solution (in milliliters), and then multiplying by 100.

As used herein the term “soluble” when having reference to a bladder therapeutic agent means that the substance is at least soluble according to the method described by L. Prosky et al, J. Assoc. Off. Anal. Chem. 71, 1017-1023 (1988).

### DETAILED DESCRIPTION OF THE DISCLOSURE

The compositions and formulations of the present disclosure are intended to stimulate the growth of Gram-positive rod-shaped bacteria belonging to the species Lactobacilli spp. It is believed that stimulating the growth of, and dominance of, lactobacillus reestablishes healthy flora by reducing or excluding the population of pathogenic bacteria. The compositions of the present disclosure generally comprises a bladder therapeutic agent capable of facilitating the growth of Gram-positive rod-shaped bacteria belonging to the species Lactobacilli spp.

The present disclosure is related to compositions and methods useful in modulating the bladder microbiome to improve bladder health, such as to treat or prevent incontinence, overactive bladder, or urinary tract infections in a subject. The compositions can be configured to be administered to a subject through topical application in various forms, including, but not limited to, a liquid, cream, gel, or spray. Compositions can be alternatively or additionally administered to a subject through a delivery mechanism such as, for example, a wipe substrate or by being applied to at least a portion of an absorbent article that can deliver the composition to the subject. For example, the composition can be applied to a top sheet of a feminine care pad, in one example. In some embodiments, the composition could be administered to a subject through a suppository. Another way the compositions may be configured to be administered to a subject can be by having the composition be configured in the form of a pill that can be ingested by the subject.

Compositions and formulations of the present disclosure are particularly well suited for administration to the urogenital region to support and maintain a healthy microflora of the bladder. For example, maintenance and support of a healthy microflora may be achieved by topically administering a composition to the urogenital area, such as the vagina. In some embodiments, the composition including a bladder therapeutic agent can be administered to the urethra or periurethral area of a subject. Generally, the bladder therapeutic agent can include a prebiotic including isomaltulose, dextrin, or combinations thereof. In some embodiments, dextrin can be dextrin type 1, dextrin type 2, or combinations thereof. Isomaltulose can be obtained from Beneo GmbH (Mannheim, Germany) under the tradename Palatinose™. Dextrin type 1 and dextrin type 2 are available from Sigma Aldrich.

Twenty-nine prebiotic compounds (Table 1) were screened for the ability to support the growth of healthy Lactobacillus species and to determine whether they did not support the growth of bacteria associated with disease (Streptococcus anginosus and Enterococcus faecalis). The twenty-nine prebiotic compounds were selected because these prebiotics have been shown in past testing to provide positive results for growing Lactobacillus species in microbiomes other than the bladder microbiome, such as the vaginal microbiome.

TABLE 1
Prebiotic Compounds

| Compound Tested | CAS # | Manufacturer | Cat/Prod No. |
|---|---|---|---|
| Alpha-D-Lactose | 5989-81-1 | Sigma | L8783-1KG |
| Maltitol | 585-88-6 | Sigma | M8892-100G |
| N-Acetylglucosamine | 7512-17-6 | MP Biomedicals, Inc (Fisher) | ICN10006880 |
| Alpha-Cyclodextrin | 10016-20-3 | Spectrum (Fisher) | 18-600-437 |
| Glucomannan | 37220-17-0 | Spectrum | G1260-25KGBL |
| Tagatose | 87-81-0 | Alfa Aesar (Fisher) | B2119206 |
| 2-Deoxy-D-Ribose | 533-67-5 | Sigma | D5899-1G |
| Alpha-Methyl-D-Glucoside | 97-30-3 | Sigma | 66940-100G |
| Amylopectin from corn | 9037-22-3 | TCI America (Fisher) | A0456500G |
| Amylopectin from potato | 9037-22-3 | Sigma | A8515-25G |
| starch | | | |
| Beta-D-Fructose | 57-48-7 | Sigma | F0127-100G |
| Beta-D-Glucose | 28905-12-6 | Spectrum | TCI-G0047-500G |
| D-Arabinose | 28697-53-2 | Fisher | BP2504-25 |
| D-Cellobiose | 528-50-7 | MP Biomedicals, Inc (Fisher) | ICN10129805 |
| Dextran Mr ~6,000 | 9004-54-0 | Sigma | 31388 |
| Dextrin, type 1 | 9004-53-9 | Sigma | D2006-500G |
| Dextrin, type 2 | 9004-53-9 | Sigma | D2131-500G |
| DL-Malic acid (Malate) | 6915-15-7 | Sigma | W265501-1KG-K |
| D-Trehalose | 6138-23-4 | Fisher | 90210-50G |
| Ecodermine ™ (4-O-.beta.-D- | N/A | Sederma | |
| Galactopyranosyl-D-glucitol | | | |
| (585-86-4), Glycerin (56-81-5), | | | |
| Xylitol (87-99-0)) | | | |
| Lactitol | 81025-04-9 | Sigma | L3520-5G |
| Lactulose | 4618-18-2 | Sigma | L7877-25G |
| Maltitol | 585-88-6 | Sigma | PHR1248-500MG |
| Maltotriose | 1109-28-0 | Alfa Aesar (Fisher) | J6649106 |
| Palatinose Hydrate ™ | 13718-94-0 | Alfa Aesar (Fisher) | AAJ6009122 |
| (isomaltulose) (6-O-α-D- | | | |
| Glucopyranosyl-D-fructose, | | | |
| Isomaltulose hydrate, isomaltose) | | | |
| Palatinose PST-PA | 13718-94-0 | Beneo-Orafti | PST-PA |
| (particle size <0.05 mm) | | | |
| Palatinose PST-PF | 13718-94-0 | Beneo-Orafti | PST-PF |
| (particle size <0.1 mm) | | | |
| Palatinose PST-N | 13718-94-0 | Beneo-Orafti | PST-N |
| (particle size <0.71 mm) | | | |
| Pectin from apple | 9000-69-5 | Sigma | 76282-100G |
| Pullulan | 9057-02-7 | MP Biomedicals, Inc (Fisher) | MP215394425 |
| Salicin | 138-52-3 | ACROS Organics (Fisher) | AC132591000 |
| Xylitol | 87-99-0 | Sigma | X3375-100G |

Most of the prebiotic compounds were initially tested in a high throughput assay screening process, described further in the Test Methods section herein, but three prebiotics (as noted in Table 3) were tested using a plate assay screening process as described in the Test Methods section herein. The prebiotics were tested against the commensal bacterial species of Lactobacillus crispatus, Lactobacillus gasseri, and Lactobacillus jensenii and against the pathogenic bacterial species of Streptococcus anginosus and Enterococcus faecalis as shown in Table 2. The bacterial species were taken from different sources, listed as culture collection, bladder, and vagina.

TABLE 2
Bacteria Species used in Assay Plate Screening

| Notation | Bacterial Species | Specimen ID | Source |
|---|---|---|---|
| Ef - 1 | E. faecalis | ATCC BAA-2128 | Culture Collection |
| Ef - 2 | E. faecalis | KC16-7171-3 | Bladder |
| Ef - 3 | E. faecalis | KC17-3969-2B | Bladder |
| Ef - 4 | E. faecalis | KC17-4331-3 | Bladder |
| Lc - 1 | L. crispatus | ATCC 33820 | Culture Collection |
| Lc - 2 | L. crispatus | KC16-7134-3C | Bladder |
| Lc - 3 | L. crispatus | KC18-1173-1 | Bladder |
| Lc - 4 | L. crispatus | KC18-1174-1 | Vagina |
| Sa - 1 | S. anginosus | ATCC 33397 | Culture Collection |
| Sa - 2 | S. anginosus | KC18-1131-3B | Bladder |
| Lg - 1 | L. gasseri | KC16-7135-1 | Bladder |
| Lg - 2 | L. gasseri | KC16-7171-1 | Bladder |
| Lg - 3 | L. gasseri | KC18-1131-2 | Bladder |
| Lg - 4 | L. gasseri | KC18-1142-2 | Bladder |
| Lj - 1 | L. jensenii | KC17-4297-18 | Bladder |
| Lj - 2 | L. jensenii | KC17-4347-1 | Bladder |
| Lj - 3 | L. jensenii | KC17-4368-2 | Bladder |

The results of the assay plate screening of the prebiotic compounds of Table 1 against the bacterial species of Table 2 is documented in Table 3 with the results being described by the following legend:
++ if growth and fermentation was equal to or greater than the positive control
+ if growth and fermentation was less than the positive control
+ if weak or no growth or fermentation
NT if not tested
   Some codes were not tested based on the use or non-use by the bacteria, as a way to efficiently move through the assay plate screening to the competition assay, described below.

TABLE 3
Prebiotic Screening Results

| | Prebiotic Compounds | | | | | | | | | | | |
|---|---|---|---|---|---|---|---|---|---|---|---|---|
| | Dextrose | CHL | | Alpha- | | | | | | | | DL- |
| Bac | (pos | (neg | 2-Deoxy- | Methyl-D- | Beta-D- | Beta-D- | D- | D- | | | | Malic |
| Spec. | control) | control) | D-Ribose | Glucoside | fructose | glucose | Arabinose | Trehalose | Ecodermine | Lactitol | Lactulose | acid |
| Ef-1 | ++ | − | − | NT | NT | ++ | NT | NT | ++ | + | NT | NT |
| Ef-2 | ++ | − | − | − | ++ | ++ | − | ++ | ++ | − | − | NT |
| Ef-3 | ++ | − | − | − | ++ | ++ | − | ++ | ++ | − | +² | NT |
| Ef-4 | ++ | − | − | − | ++ | ++ | − | ++ | ++ | − | +² | NT |
| Lc-1 | ++ | − | − | NT | NT | ++ | NT | NT | − | − | NT | NT |
| Lc-2 | ++ | − | − | + | ++ | ++ | − | NT | − | − | − | − |
| Lc-3 | ++ | − | − | − | ++ | ++ | − | ++ | − | − | − | − |
| Lc-4 | ++ | − | − | + | ++ | ++ | − | + | − | − | ++ | − |
| Sa-1 | ++ | − | − | NT | NT | ++ | NT | NT | − | − | NT | NT |
| Sa-2 | ++ | − | − | − | ++ | ++ | − | ++ | − | − | ++ | NT |
| Lg-1 | ++ | − | − | − | + | ++ | − | ++ | − | − | − | − |
| Lg-2 | ++ | − | − | − | + | ++ | − | ++ | − | − | − | − |
| Lg-3 | ++ | − | NT | − | + | NT | − | ++ | NT | NT | − | − |
| Lg-4 | ++ | − | NT | − | + | NT | − | − | NT | NT | + | NT |
| Lj-1 | ++ | − | NT | − | ++ | NT | NT | + | NT | NT | − | NT |
| Lj-2 | ++ | − | − | + | + | − | − | − | − | NT | − | − |
| Lj-3 | ++ | − | NT | NT | ++ | NT | NT | NT | NT | NT | − | NT |

| | Prebiotic Compounds | | | | | | | | |
|---|---|---|---|---|---|---|---|---|---|
| Bac | | | N- | | | | Palatinose | | Amylopectin |
| Spec. | Maltitol | Maltotriose | Acetylglucosamine | Salicin | Tagatose | Xylitol | (isomaltulose) | Pullulan | corn |
| Ef-1 | − | ++ | ++ | ++ | ++ | − | NT | NT | NT |
| Ef-2 | − | ++ | ++ | ++ | ++ | − | − | − | +² |
| Ef-3 | − | ++ | ++ | ++ | ++ | − | − | − | +² |
| Ef-4 | − | ++ | ++ | ++ | ++ | − | − | − | − |
| Lc-1 | − | ++ | ++ | ++ | − | − | NT | NT | NT |
| Lc-2 | − | ++ | + | − | − | − | − | NT | NT |
| Lc-3 | − | ++ | ++ | − | − | − | ++ | − | − |
| Lc-4 | − | ++ | + | − | − | − | ++ | NT | NT |
| Sa-1 | − | ++ | ++ | ++ | − | − | NT | NT | NT |
| Sa-2 | − | ++ | ++ | ++ | − | − | − | − | − |
| Lg-1 | − | ++ | ++ | ++ | − | − | − | − | − |
| Lg-2 | − | ++ | ++ | − | − | − | NT | NT | NT |
| Lg-3 | NT | NT | NT | NT | NT | NT | NT | NT | NT |
| Lg-4 | NT | NT | NT | NT | NT | NT | NT | NT | NT |
| Lj-1 | NT | NT | NT | NT | NT | NT | + | − | − |
| Lj-2 | − | ++ | − | ++ | − | − | NT | NT | NT |
| Lj-3 | NT | NT | NT | NT | NT | NT | NT | NT | NT |

| | Prebiotic Compounds | | | | | | | |
|---|---|---|---|---|---|---|---|---|
| | | | Dextrin | Dextrin | | | | |
| Bac | Amylopectin | Dextran | Type | Type | Alpha | | | D- |
| Spec. | potato | (MW~6,000)¹ | 1 | 2 | Cyclodextrin¹ | Glucomannan¹ | Pectin¹ | Cellobiose |
| Ef-1 | NT | NT | NT | NT | NT | NT | NT | NT |
| Ef-2 | +² | NT | + | +² | + | ++ | +² | ++ |
| Ef-3 | +² | NT | + | +² | NT | NT | NT | ++ |
| Ef-4 | − | NT | + | +² | NT | NT | NT | ++ |
| Lc-1 | − | + | NT | NT | NT | NT | NT | NT |
| Lc-2 | NT | + | NT | NT | + | NT | − | ++ |
| Lc-3 | − | + | + | + | +² | + | ++ | + |
| Lc-4 | NT | NT | NT | NT | NT | NT | NT | + |
| Sa-1 | NT | NT | NT | NT | NT | NT | NT | NT |
| Sa-2 | − | NT | + | +² | + | ++ | +² | ++ |
| Lg-1 | − | NT | ++ | + | − | + | +² | + |
| Lg-2 | NT | NT | NT | NT | NT | NT | NT | ++ |
| Lg-3 | NT | NT | NT | NT | NT | NT | NT | ++ |
| Lg-4 | NT | NT | NT | NT | NT | NT | NT | ++ |
| Lj-1 | − | NT | + | + | + | + | +² | + |
| Lj-2 | NT | NT | NT | NT | NT | NT | NT | − |
| Lj-3 | NT | NT | NT | NT | NT | NT | NT | NT |

¹Plate assay performed due to prebiotics interfering with spectrophotometer absorbency reads.
²Bacteria demonstrated growth but no fermentation on the prebiotic

As noted from Table 3, some of the prebiotic compounds were not able to provide growth for most, if not all, commensal and pathogenic bacterial species from a culture collection or the bladder. Exemplary prebiotic compounds providing these results were 2-Deoxy-D-Ribose, D-Arabinose, lactitol, DL-Malic Acid, maltitol, xylitol, and pullulan. Some of the results were surprising, such as the results for lactitol, which is a known vaginal prebiotic. The test results for lactitol did not show any positive growth for commensal bacterial species from culture collection, the bladder, or the vagina. Thus, these prebiotic compounds do not appear to have much promise for modulating the bladder microbiome to improve bladder health.

Table 3 also documents that some of the prebiotic compounds were able to provide growth on the commensal bacterial species, but also provided growth on the pathogenic bacterial species. Exemplary prebiotic compounds providing these results were beta-D-fructose, beta-D-glucose, maltotriose, N-Acetylglucosamine, salicin, and D-Cellobiose. Some compounds provided weak or no growth on commensal bacteria, but provided growth on pathogenic bacteria. An exemplary prebiotic exhibiting these results was ecodermine. Such compounds do not provide much promise for modulating the bladder microbiome in a positive manner to improve bladder health.

However, Table 3 also documents that several prebiotic compounds showed promise as growing at least some of the commensal bacterial species and either having less growth than the positive control or almost no growth of the pathogenic bacterial species compared to the control. Exemplary prebiotic compounds fitting in this category that showed initial promise include: alpha-methyl-D-glucoside, isomaltulose, dextrin type 1, dextrin type 2, alpha cyclodextrin, and pectin.

Additional screening of three prebiotic compounds of alpha-methyl-D-glucoside, isomaltulose, and alpha cyclodextrin was completed against pathogenic bacteria species Escherichia coli from both bladder and vaginal sources, as shown in Table 4. The performance of these prebiotic compounds against E. coli strains is shown in Table 5. The growth of the bacterial species for this additional screening process against E. coli was not compared to growth of a control as in the screening process documented in Table 3, but was documented based on growth or no growth for the number of samples tested.

TABLE 4
Additional Bacteria Species used in Screening

| Notation | Bacterial Species | Specimen ID | Source |
|---|---|---|---|
| Ec-1 | E. coli | KC16-7171-8 | Bladder |
| Ec-2 | E. coli | KC17-3969-1 | Bladder |
| Ec-3 | E. coli | KC17-3970-4 | Vagina |
| Ec-4 | E. coli | KC17-4296-4 | Vagina |
| Ec-5 | E. coli | KC17-4297-5 | Bladder |

TABLE 5
Additional Prebiotic Screening Results

| | | Additional Bacterial Screening Species | | | | |
|---|---|---|---|---|---|---|
| Prebiotic Compounds | # | Ec-1 | Ec-2 | Ec-3 | Ec-4 | Ec-5 |
| Alpha-Methyl-D-Glucoside | # grew | 0 | 0 | 0 | 0 | 0 |
| | # fermented | 0 | 0 | 0 | 0 | 0 |
| | # tested | 2 | 2 | 2 | 2 | 2 |
| Palatinose (isomaltulose) | # grew | 0 | 0 | 0 | 0 | 0 |
| | # fermented | 0 | 0 | 0 | 0 | 0 |
| | # tested | 2 | 2 | 2 | 2 | 2 |
| Alpha Cyclodextrin¹ | # grew | NT | NT | 1 | NT | NT |
| | # fermented | NT | NT | 0 | NT | NT |
| | # tested | NT | NT | 1 | NT | NT |

¹Plate assay performed due to prebiotics interfering with spectrophotometer absorbency reads.
NT = Not Tested

As documented in Table 5, Alpha-Methyl-D-Glucoside as well as isomaltulose (palatinose) did not grow or ferment the pathogenic bacterial species of E. coli, regardless of the source of the E. coli (either vaginal or bladder). However, Alpha Cyclodextrin did grow the one E. coli species tested (Ec-3) that was from a vaginal source.

Further testing was conducted to put the prebiotic compounds in competition assay testing, as further described in the test methods section herein. In the competition assay testing, each competition involved testing a single prebiotic compound with the commensal bacteria species of L. crispatus, KC18-1173-1, Bladder (“Lc-3”—Table 2) against the pathogenic bacterial species of S. anginosus, KC18-1131-3B, Bladder (“Sa-2”—Table 2). The results of the competition assay testing are shown in Tables 6 and 7.

TABLE 6
Competition Assay Testing

| Competition | | | Log CFU/mL | | | | | |
|---|---|---|---|---|---|---|---|---|
| Bacterial | | | | MRS | MRS | TSA | TSA | Therapeutic |
| Species | Prebiotic | Replicate | Start | end | Δ | end | Δ | Effect |
| Sa-2 | Palatinose | A | 5.49 | 4.47 | −1.90 | 4.56 | −2.07 | 2.57E+03 |
| | Hydrate (2%) | B | 5.38 | NA | NA | 5.73 | −0.79 | 3.29E+03 |
| | | C | 6.15 | 4.88 | −1.82 | 4.53 | −2.32 | 6.92E+03 |
| | | D | 6.90 | 5.16 | −1.69 | 4.74 | −2.39 | 1.74E+03 |
| Lc-3 | | A | 3.80 | 7.97 | 2.93 | ND | | 2.57E+03 |
| | | B | 4.80 | 8.00 | 2.26 | ND | | 3.29E+03 |
| | | C | 5.22 | 8.37 | 3.24 | ND | | 6.92E+03 |
| | | D | 5.10 | 7.98 | 1.90 | ND | | 1.74E+03 |
| Sa-2 | Palatinose | A | 5.49 | NA | NA | 6.07 | −0.56 | 1.66E+02 |
| | PST-PA (2%) | B | 5.38 | 5.06 | −1.63 | 5.29 | −1.23 | 1.50E+02 |
| Lc-3 | | A | 3.80 | 8.29 | 3.25 | ND | | 1.66E+02 |
| | | B | 4.80 | 7.68 | 1.94 | ND | | 1.50E+02 |
| Sa-2 | Palatinose | A | 5.49 | NA | NA | 3.95 | −2.68 | 1.29E+04 |
| | PST-PF (2%) | B | 5.38 | 5.41 | −1.28 | 5.58 | −0.94 | 1.41E+04 |
| Lc-3 | | A | 3.80 | 8.06 | 3.02 | ND | | 1.29E+04 |
| | | B | 4.80 | 7.90 | 2.16 | ND | | 1.41E+04 |
| Sa-2 | Palatinose | A | 5.49 | 4.29 | −2.08 | 4.52 | −2.11 | 8.71E+03 |
| | PST-N (2%) | B | 5.38 | 5.16 | −1.53 | 5.49 | −1.03 | 5.80E+03 |
| Lc-3 | | A | 3.80 | 8.46 | 3.42 | ND | | 8.71E+03 |
| | | B | 4.80 | 8.22 | 2.48 | ND | | 5.80E+03 |
| Sa-2 | Lactitol (2%) | B | 6.15 | 7.45 | 0.75 | 7.43 | 0.58 | 2.95E−02 |
| | | C | 6.90 | 7.54 | 0.69 | 7.65 | 0.52 | 1.23E−01 |
| Lc-3 | | B | 5.22 | 5.90 | 0.77 | ND | | 2.95E−02 |
| | | C | 5.10 | 6.74 | 0.66 | ND | | 1.23E−01 |
| Sa-2 | 2-Deoxy-D- | B | 6.15 | 4.29 | −2.41 | 6.22 | −0.63 | 6.03E−07 |
| | ribose (2%) | C | 6.90 | 5.59 | −1.26 | 6.90 | −0.23 | 1.91E−03 |
| Lc-3 | | B | 5.22 | 0 | −5.22 | ND | | 6.03E−07 |
| | | C | 5.1 | 4.18 | −1.90 | ND | | 1.91E−03 |
| Sa-2 | D-Arabinose | B | 6.15 | 6.43 | −0.27 | 6.39 | −0.46 | 4.07E−02 |
| | (2%) | C | 6.90 | 6.38 | −0.47 | 6.81 | −0.32 | 1.10E−01 |
| Lc-3 | | B | 5.22 | 5.00 | −0.13 | ND | | 4.07E−02 |
| | | C | 5.10 | 5.85 | −0.23 | ND | | 1.10E−01 |
| Sa-2 | Maltitol (2%) | B | 6.15 | 6.58 | −0.12 | 7.26 | 0.41 | 1.66E−02 |
| | | C | 6.9 | 6.48 | −0.37 | 7.6 | 0.47 | 2.51E−02 |
| Lc-3 | | B | 5.22 | 5.48 | 0.35 | ND | | 1.66E−02 |
| | | C | 5.1 | 6.00 | −0.08 | ND | | 2.51E−02 |
| Sa-2 | alpha-Methyl-D- | B | 6.15 | 7.15 | 0.45 | 7.47 | 0.62 | 1.21E−01 |
| | glucoside (2%) | C | 6.90 | 7.40 | 0.55 | 7.54 | 0.41 | 1.74E−01 |

| | | | | | | |
|---|---|---|---|---|---|---|
| Lc-3 | B | 5.22 | 6.52 | 1.39 | ND | 1.21E−01 |
| | C | 5.10 | 6.78 | 0.70 | ND | 1.74E−01 |

¹Background growth for replicate A, Sa-2 background growth in CHL media only quantified on MRS agar 0.88 Log CFU/mL, quantified on TSA 1.14 Log CFU/mL. Lc-3 background growth in CHL media only quantified on MRS agar 1.24 Log CFU/mL.
²Background growth for replicate B, Sa-2 background growth in CHL media only quantified on MRS agar 1.31 Log CFU/mL, quantified on TSA 1.14 Log CFU/mL. Lc-3 background growth in CHL media only quantified on MRS agar 1.44 Log CFU/mL.
³Background growth for replicate C, Sa-2 background growth in CHL media only quantified on MRS agar 0.55 Log CFU/mL, quantified on TSA 0.70 Log CFU/mL. Lc-3 background growth in CHL media only quantified on MRS agar −0.09 Log CFU/mL.
⁴Background growth for replicate D, Sa-2 background growth in CHL media only quantified on MRS agar −0.05 Log CFU/mL, quantified on TSA 0.23 Log CFU/mL. Lc-3 background growth in CHL media only quantified on MRS agar 0.98 Log CFU/mL.
⁵ND, not determined. TSA does not support the growth of Lactobacillus crispatus.

TABLE 7
Additional Competition Assay Testing

| Competition | | | Log CFU/mL | | | | | |
|---|---|---|---|---|---|---|---|---|
| Bacterial | | | | MRS | MRS | TSA | TSA | Therapeutic |
| Species | Prebiotic | Replicate | Start | end | Δ | end | Δ | Effect |
| Sa-2 | Dextrin Type | A | 6.54 | 0.00 | (−6.54) | 2.74 | (−3.80) | 1.25E+05 |
| | 1 (2%) | B | 6.70 | 4.85 | (−1.86) | 4.90 | (−1.80) | 2.14E+03 |
| Lc-3 | | A | 4.78 | 7.82 | 2.82 | ND | | 1.25E+05 |
| | | B | 4.56 | 8.23 | 3.33 | ND | | 2.14E+03 |
| Sa-2 | Dextrin Type | A | 6.54 | 0.00 | (−6.54) | 2.81 | (−3.73) | 3.98E+05 |
| | 2 (2%) | B | 6.70 | 6.65 | (−0.05) | 6.65 | (−0.05) | 8.12E+00 |
| Lc-3 | | A | 4.78 | 8.41 | 2.67 | ND | | 3.98E+05 |
| | | B | 4.56 | 7.56 | 2.65 | ND | | 8.12E+00 |
| Sa-2 | Glucomannan | A | 6.54 | 7.45 | 0.91 | 7.59 | 1.05 | 1.82E+01 |
| Lc-3 | (0.5%) | A | 4.78 | 6.85 | 1.70 | ND | | 1.82E+01 |
| Sa-2 | Pectin | A | 6.54 | 7.51 | 0.97 | 7.55 | 1.01 | 2.82E−04 |
| Lc-3 | (Apple) | A | 4.78 | 4.0 | (−1.15) | ND | | 2.82E−04 |
| Sa-2 | Amylopectin | B | 6.70 | 7.37 | 0.67 | 7.41 | 0.71 | 2.34E−02 |

| | | | | | | | |
|---|---|---|---|---|---|---|---|
| Lc-3 | (Potato) | B | 4.56 | 5.78 | 0.88 | ND | 2.34E−02 |

¹Background growth in CHL media alone for Replicate A was subtracted from all bacterial growth. Lc-3 had 0.37 Log CFU/mL background growth subtracted. No background growth was observed for Sa-2 recovered on MRS or TSA.
²Background growth in CHL media alone for Replicate B was subtracted from all bacterial growth. Lc-3 had 0.34 Log CFU/mL background growth subtracted. No background growth was observed for Sa-2 recovered on MRS or TSA.

Tables 6 and 7 demonstrate that when tested in a bacterial competition assay, only three of the prebiotics tested of the twenty-nine prebiotics selected supported the growth of Lactobacillus crispatus over Streptococcus anginosus. Thus, the prebiotics that showed the most promise as bladder therapeutic agents include isomaltulose and dextrin (such as dextrin type 1, and dextrin type 2).

Accordingly, in a preferred embodiment, the composition comprising a bladder therapeutic agent including isomaltulose, dextrin type 1, dextrin type 2, or combinations thereof effects the growth of L. crispatus over S. anginosus in the bladder, as measured using the therapeutic effect protocol described below. Preferably the composition yields therapeutic effect (a ratio of L. crispatus to S. anginosus) in the bladder greater than about 100, still more preferably greater than about 500 and still more preferably greater than 1,000, and even more preferably greater than about 5,000. In some embodiments, the composition can yield a therapeutic effect greater than 10,000.

Surprisingly, compositions comprising a bladder therapeutic agent including isomaltulose, dextrin type 1, dextrin type 2, and combinations thereof can promote the growth of healthy bacteria such as Lactobacillus spp. and more particularly Lactobacillus crispatus in the bladder without promoting growth of enteropathogenic bacteria, such as Streptococcus anginosus in the bladder. As noted above, this result is surprising from the standpoint that various other prebiotic compositions were not able modulate the bladder microbiome in this manner even though they may have been expected to because these prebiotics were previously known to provide a positive effect on commensal bacteria from the vaginal microbiome with either a neutral or inhibiting effect on pathogenic bacteria from the vaginal microbiome.

Bladder treatment compositions of the present disclosure can be administered in several forms to a user. For example, the bladder treatment compositions may be prepared as formulations for administration to a user or may be applied to a substrate, such as a wiping substrate, for administration to a user. Preferably the bladder therapeutic agents useful in the present disclosure are soluble to facilitate their formulation for administration to a user.

The bladder therapeutic agents should be provided in an amount sufficient to provide a therapeutic effect when administered to a subject. For example, where the composition comprises a bladder therapeutic agent comprising isomaltulose, dextrin type 1, dextrin type 2, and combinations thereof, the bladder therapeutic agent is present in an amount sufficient to stimulate the growth of certain healthy bladder bacteria such as Lactobacillus crispatus, Lactobacillus gasseri, and Lactobacillus jensenii. Preferably the composition provides a therapeutic effect, measured as the ratio of L. crispatus to S. anginosus as described in the test methods section below, of greater than about 100, more preferably greater than about 500, and more preferably greater than about 1,000, and even more preferably greater than about 5,000.

In some embodiments, compositions of the present disclosure comprise less than about 10.0 wt/vol % of bladder therapeutic agent. In some embodiments the total amount of therapeutic agent is less than about 7.5 wt/vol %, or less than about 5.0 wt/vol %, such as from about 0.01 to about 2.5 wt/vol %, or from about 0.1 to about 1.5 wt/vol %. For example, in one embodiment, the composition comprises from about 0.1 to about 2.0 wt/vol % of a bladder therapeutic agent comprising isomaltulose, dextrin type 1, dextrin type 2, and combinations thereof.

The compositions of the present disclosure may be formulated for administration to a user. For example, in those embodiments where the composition is formulated as a bladder treatment formulation, it may be formulated as a spray, moisturizer, lotion, cream, jelly, liniment, ointment, salve, oil, foam, gel, film, wash, suppository, slow-releasing polymer, coating, liquid, vaginal capsule, vaginal tablet, vaginal film, vaginal sponge, vaginal ovule, etc. The composition may also be applied to a vaginal insert, tampon, wipe or pad, and then administered to the vagina.

Compositions including a bladder therapeutic agent can include a “dermatologically acceptable carrier”, which refers to a carrier that is suitable for topical application to the keratinous tissue and is compatible with the bladder therapeutic agent. The dermatologically acceptable carrier may be in a wide variety of forms such as, for example, simple solutions (water-based or oil-based), solid forms (e.g. gels or sticks) and emulsions. In some embodiments, the carrier can be either aqueous or non-aqueous.

Water is a particularly preferred aqueous carrier. Non-aqueous carriers may include, for example, glycols, such as propylene glycol, butylene glycol, triethylene glycol, hexylene glycol, polyethylene glycols, ethoxydiglycol, and dipropyleneglycol; alcohols, such as ethanol, n-propanol, and isopropanol; triglycerides; ethyl acetate; acetone; triacetin; and combinations thereof. In some embodiments, the carrier constitutes greater than about 75 wt/vol %, more preferably greater than about 85 wt/vol %, and still more preferably greater than about 90 wt/vol %. In some embodiments, the carrier can constitute greater than about 95 wt/vol. %, or greater than about 96 wt/vol. %, 97 wt/vol. %, 98 wt/vol. %, or even 99 wt/vol. %.

The composition may include other components such as, for example, a surfactant, an ester, a humectant, a pH adjuster, a rheology modifier, a gelling agent, and/or an antimicrobial agent.

Surfactant

In some embodiments, the composition can include one or more surfactants. In an embodiment where the composition is included in a wipe, the composition may also likely include one or more surfactants. These may be selected from anionic, cationic, nonionic, zwitterionic, and amphoteric surfactants. Amounts of surfactants may range from 0.01 to 30%, or from 10 to 30%, or from 0.05 to 20%, or from 0.10 to 15% by total weight of the composition. In some embodiments, such as when the wetting composition is used with a wipe, the surfactant can comprise less than 5% by total weight of the wetting composition.

Suitable anionic surfactants include, but are not limited to, C₈ to C₂₂ alkane sulfates, ether sulfates and sulfonates. Among the suitable sulfonates are primary C₈ to C₂₂ alkane sulfonate, primary C₈ to C₂₂ alkane disulfonate, C₈ to C₂₂ alkene sulfonate, C₈ to C₂₂ hydroxyalkane sulfonate or alkyl glyceryl ether sulfonate. Specific examples of anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, potassium lauryl sulfate, sodium trideceth sulfate, sodium methyl lauroyl taurate, sodium lauroyl isethionate, sodium laureth sulfosuccinate, sodium lauroyl sulfosuccinate, sodium tridecyl benzene sulfonate, sodium dodecyl benzene sulfonate, sodium lauryl amphoacetate and mixtures thereof. Other anionic surfactants include the C₈ to C₂₂ acyl glycinate salts. Suitable glycinate salts include sodium cocoylglycinate, potassium cocoylglycinate, sodium lauroylglycinate, potassium lauroylglycinate, sodium myristoylglycinate, potassium myristoylglycinate, sodium palmitoylglycinate, potassium palmitoylglycinate, sodium stearoylglycinate, potassium stearoylglycinate, ammonium cocoylglycinate and mixtures thereof. Cationic counter-ions to form the salt of the glycinate may be selected from sodium, potassium, ammonium, alkanolammonium and mixtures of these cations.

Suitable cationic surfactants include, but are not limited to alkyl dimethylamines, alkyl amidopropylamines, alkyl imidazoline derivatives, quaternised amine ethoxylates, and quaternary ammonium compounds.

Suitable nonionic surfactants include, but are not limited to, alcohols, acids, amides or alkyl phenols reacted with alkylene oxides, especially ethylene oxide either alone or with propylene oxide. Specific nonionics are C₆ to C₂₂ alkyl phenols-ethylene oxide condensates, the condensation products of C₈ to C₁₃ aliphatic primary or secondary linear or branched alcohols with ethylene oxide, and products made by condensation of ethylene oxide with the reaction products of propylene oxide and ethylenediamine. Other nonionics include long chain tertiary amine oxides, long chain tertiary phosphine oxides and dialkyl sulphoxides, alkyl polysaccharides, amine oxides, block copolymers, castor oil ethoxylates, ceto-oleyl alcohol ethoxylates, ceto-stearyl alcohol ethoxylates, decyl alcohol ethoxylates, dinonyl phenol ethoxylates, dodecyl phenol ethoxylates, end-capped ethoxylates, ether amine derivatives, ethoxylated alkanolamides, ethylene glycol esters, fatty acid alkanolamides, fatty alcohol alkoxylates, lauryl alcohol ethoxylates, mono-branched alcohol ethoxylates, natural alcohol ethoxylates, nonyl phenol ethoxylates, octyl phenol ethoxylates, oleyl amine ethoxylates, random copolymer alkoxylates, sorbitan ester ethoxylates, stearic acid ethoxylates, stearyl amine ethoxylates, synthetic alcohol ethoxylates, tall oil fatty acid ethoxylates, tallow amine ethoxylates and trid tridecanol ethoxylates.

Suitable zwitterionic surfactants include, for example, alkyl amine oxides, alkyl hydroxysultaines, silicone amine oxides, and combinations thereof. Specific examples of suitable zwitterionic surfactants include, for example, 4-[N,N-di(2-hydroxyethyl)-N-octadecylammonio]-butane-1-carboxylate, S-[S-3-hydroxypropyl-S-hexadecylsulfonio]-3-hydroxypentane-1-sulfate, 3-[P,P-diethyl-P-3,6,9-trioxatetradexopcylphosphonio]-2-hydroxypropane-1-phosphate, 3-[N,N-dipropyl-N-3-dodecoxy-2-hydroxypropylammonio]-propane-1-phosphonate, 3-(N,N-dimethyl-N-hexadecylammonio)propane-1-sulfonate, 3-(N,N-dimethyl-N-hexadecylammonio)-2-hydroxypropane-1-sulfonate, 4-[N,N-di(2-hydroxyethyl)-N-(2-hydroxydodecyl)ammonio]-butane-1-carboxylate, 3-[S-ethyl-S-(3-dodecoxy-2-hydroxypropyl)sulfonio]-propane-1-phosphate, 3-[P,P-dimethyl-P-dodecylphosphonio]-propane-1-phosphonate, 5-[N,N-di(3-hydroxypropyl)-N-hexadecylammonio]-2-hydroxy-pentane-1-sulfate, lauryl hydroxysultaine and combinations thereof.

Suitable amphoteric surfactants include, but are not limited to, derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one substituent contains an anionic group, e.g., carboxy, sulfonate, sulfate, phosphate, or phosphonate. Illustrative amnphoterics are coco dimethyl carboxymethyl betaine, cocoamidopropyl betaine, cocobetaine, oleyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, cocoamphoacetates, and combinations thereof. The sulfobetaines may include stearyl dimethyl sulfopropyl betaine, lauryl dimethyl sulfoethyl betaine, lauryl bis-(2-hydroxyethyl) sulfopropyl betaine and combinations thereof.

Esters

In some embodiments, the compositions include one or more esters. The esters may be selected from cetyl palmitate, stearyl palmitate, cetyl stearate, isopropyl laurate, isopropyl myristate, isopropyl palmitate, and combinations thereof. The fatty alcohols include octyldodecanol, lauryl, myristyl, cetyl, stearyl, behenyl alcohol, and combinations thereof. The fatty acids can include, but are not limited to, capric acid, undecylenic acid, lauric acid, Myristic acid, palmitic acid, stearic acid, oleic acid, linoleic acid, arachidic acid, and behenic acid. Ethers such as eucalyptol, ceteraryl glucoside, dimethyl isosorbic polyglyceryl-3 cetyl ether, polyglyceryl-3 decyltetradecanol, propylene glycol myristyl ether, and combinations thereof can also suitably be used as emollients. Other suitable ester compounds for use in the antimicrobial compositions or the present disclosure are listed in the International Cosmetic Ingredient Dictionary and Handbook, 11th Edition, CTFA, (January, 2006) ISBN-10: 1882621360, ISBN-13: 978-1882621361, and in the 2007 Cosmetic Bench Reference, Allured Pub. Corporation (Jul. 15, 2007) ISBN-10: 1932633278, IBN-13: 978-1932633276, both of which are incorporated by reference herein to the extent they are consistent herewith.

Humectants

Humectants that are suitable as carriers in the compositions of the present disclosure include, for example, glycerin, glycerin derivatives, hyaluronic acid, hyaluronic acid derivatives, betaine, betaine derivatives, amino acids, amino acid derivatives, glycosaminoglycans, glycols, polyols, sugars, sugar alcohols, hydrogenated starch hydrolysates, hydroxy acids, hydroxy acid derivatives, salts of PCA and the like, and combinations thereof. Specific examples of suitable humectants include honey, sorbitol, hyaluronic acid, sodium hyaluronate, betaine, lactic acid, citric acid, sodium citrate, glycolic acid, sodium glycolate, sodium lactate, urea, propylene glycol, butylene glycol, pentylene glycol, ethoxydiglycol, methyl gluceth-10, methyl gluceth-20, polyethylene glycols (as listed in the International Cosmetic Ingredient Dictionary and Handbook such as PEG-2 through PEG 10), propanediol, xylitol, maltitol, or combinations thereof.

The compositions of the disclosure may include one or more humectants in an amount of about 0.01% (by total weight of the composition) to about 20% (by total weight of the composition), or about 0.05% (by total weight of the composition) to about 10% by total weight of the composition), or about 0.1% (by total weight of the composition) to about 5.0% (by total weight of the composition).

pH Adjusting Agent

In some embodiments, the compositions of the present disclosure can be acidic, i.e., have a pH less than about 7.0 and more preferably less than about 6.0, such as from about 3.0 to about 6.0 and still more preferably from about 4.0 to about 5.0. In a particularly preferred embodiment, the pH may be maintained at a mildly acidic level to correspond to normal vaginal conditions, the environment in which the composition will typically be delivered. For example, the pH may be within a range of from about 3.0 to about 6.0, in some embodiments from about 3.5 to about 5.0, and in some embodiments, from about 4.0 to about 4.5. The foregoing acid pH may also provide other benefits. For instance, when the composition is configured to form a gel, such as described below, a low pH level may also improve the gelation rate and gel strength to reduce the likelihood of leakage just after insertion of the composition into the vagina.

The pH of the composition may be adjusted using an organic acid. Organic acids useful in the present disclosure generally consist of mono- or polycarboxylic acids having one or more hydroxyl functional groups at least one of which is introduced into the α-position (i.e., on the carbon atom adjacent to the carboxyl functional group). Examples of particularly useful organic acids can include citric acid, lactic acid, methyllactic acid, phenyllactic acid, malic acid, mandelic acid, glycolic acid, tartronic acid, tartaric acid and gluconic acid. In particularly preferred embodiments the organic acid is selected from the group consisting of citric acid, lactic acid, malic acid, glycolic acid and tartaric acid. In certain embodiments the organic acid may be provided with an appropriate counterion, such as calcium, sodium or magnesium.

In view of the foregoing, in certain embodiments the compositions and formulations of the present disclosure may have a pH from about 3.0 to about 6.0, more preferably from about 3.5 to about 5.0, and comprise a bladder therapeutic agent comprising isomaltulose, dextrin type 1, dextrin type 2, and combinations thereof, wherein the total amount of bladder therapeutic agent is from about 0.1 to about 2.0 wt/vol %.

Rheology Modifier

Optionally, one or more rheology modifiers, such as thickeners, may be added to the composition. Suitable rheology modifiers are compatible with the bladder therapeutic agent. As used herein, “compatible” refers to a compound that, when mixed with the bladder therapeutic agent, does not adversely affect the properties of the bladder therapeutic agent.

A thickening system is used in the compositions to adjust the viscosity and stability of the compositions. Specifically, thickening systems prevent the composition from running off of the hands or body during dispensing and use of the composition. When the composition is used with a wipe product, a thicker formulation can be used to prevent the composition from migrating from the wipe substrate.

The thickening system should be compatible with the compounds used in the present disclosure; that is, the thickening system, when used in combination with the bladder therapeutic agent, should not precipitate out, form a coacervate, or prevent a user from perceiving the conditioning benefit (or other desired benefit) to be gained from the composition. The thickening system may include a thickener which can provide both the thickening effect desired from the thickening system and a conditioning effect to the user.

Thickeners may include, cellulosics, gums, acrylates, starches and various polymers. Suitable examples include but are not limited to hydroxethyl cellulose, xanthan gum, guar gum, potato starch, and corn starch. In some embodiments, PEG-150 stearate, PEG-150 distearate, PEG-175 diisostearate, polyglyceryl-10 behenate/eicosadioate, disteareth-100 IPDI, polyacrylamidomethylpropane sulfonic acid, butylated PVP, and combinations thereof may be suitable.

While the viscosity of the compositions will typically depend on the thickener used and the other components of the compositions, the thickeners of the compositions suitably provide for a composition having a viscosity in the range of greater than 1 cP to about 30,000 cP or more. In another embodiment, the thickeners provide compositions having a viscosity of from about 100 cP to about 20,000 cP. In yet another embodiment, thickeners provide compositions having a viscosity of from about 200 cP to about 15,000 cP. In embodiments where the compositions are included in a wipe, the viscosity may range from about 1 cP to about 2000 cP. In some embodiments, it is preferable to have a viscosity of the composition be less than 500 cP.

When including a thickening system, the compositions of the present disclosure can include the thickening system in an amount of no more than about 20% (by total weight of the composition), or from about 0.01% (by total weight of the composition) to about 20% (by total weight of the composition). In another aspect the thickening system is present in the antimicrobial composition in an amount of from about 0.10% (by total weight of the composition) to about 10% (by total weight of the composition), or from about 0.25% (by total weight of the composition) to about 5% (by total weight of the composition), or from about 0.5% (by total weight of the composition) to about 2% (by total weight of the composition).

In one embodiment, the compositions may include hydrophobic and hydrophilic ingredients, such as a lotion or cream. Generally, these emulsions have a dispersed phase and a continuous phase, and are generally formed with the addition of a surfactant or a combination of surfactants with varying hydrophilic/lipophilic balances (HLB). Suitable emulsifiers include surfactants having HLB values from 0 to 20, or from 2 to 18. Suitable non-limiting examples include Ceteareth-20, Cetearyl Glucoside, Ceteth-10, Ceteth-2, Ceteth-20, Cocamide MEA, Glyceryl Laurate, Glyceryl Stearate, PEG-100 Stearate, Glyceryl Stearate, Glyceryl Stearate SE, Glycol Distearate, Glycol Stearate, Isosteareth-20, Laureth-23, Laureth-4, Lecithin, Methyl Glucose Sesquistearate, Oleth-10, Oleth-2, Oleth-20, PEG-100 Stearate, PEG-20 Almond Glycerides, PEG-20 Methyl Glucose Sesquistearate, PEG-25 Hydrogenated Castor Oil, PEG-30 Dipolyhydroxystearate, PEG-4 Dilaurate, PEG-40 Sorbitan Peroleate, PEG-60 Almond Glycerides, PEG-7 Olivate, PEG-7 Glyceryl Cocoate, PEG-8 Dioleate, PEG-8 Laurate, PEG-8 Oleate, PEG-80 Sorbitan Laurate, Polysorbate 20, Polysorbate 60, Polysorbate 80, Polysorbate 85, Propylene Glycol Isostearate, Sorbitan Isostearate, Sorbitan Laurate, Sorbitan Monostearate, Sorbitan Oleate, Sorbitan Sesquioleate, Sorbitan Stearate, Sorbitan Trioleate, Stearamide MEA, Steareth-100, Steareth-2, Steareth-20, Steareth-21. The compositions can further include surfactants or combinations of surfactants that create liquid crystalline networks or liposomal networks. Suitable non-limiting examples include OLIVEM 1000 (INCI: Cetearyl Olivate (and) Sorbitan Olivate (available from HallStar Company (Chicago, IL)); ARLACEL LC (INCI: Sorbitan Stearate (and) Sorbityl Laurate, commercially available from Croda (Edison, NJ)); CRYSTALCAST MM (INCI: Beta Sitosterol (and) Sucrose Stearate (and) Sucrose Distearate (and) Cetyl Alcohol (and) Stearyl Alcohol, commercially available from MMP Inc. (South Plainfield, NJ)); UNIOX CRISTAL (INCI: Cetearyl Alcohol (and) Polysorbate 60 (and) Cetearyl Glucoside, commercially available from Chemyunion (Sáo Paulo, Brazil)). Other suitable emulsifiers include lecithin, hydrogenated lecithin, lysolecithin, phosphatidylcholine, phospholipids, and combinations thereof.

Gelling Agents

In some embodiments in which the composition is in the form of a gel, the disperse phase of the gel may be formed from any of a variety of different gelling agents, including temperature responsive (“thermogelling”) compounds, ion responsive compounds, and so forth. Thermogelling systems, for instance, respond to a change in temperature (e.g., increase in temperature) by changing from a liquid to a gel. Generally speaking, the temperature range of interest is from about 25° C. to about 40° C., in some embodiments from about 35° C. to about 39° C., and in one particular embodiment, at the human body temperature (about 37° C.). In some cases, thermogelling block copolymers, graft copolymers, and/or homopolymers may be employed. For example, polyoxyalkylene block copolymers may be used in some embodiments of the present invention to form a thermo-gelling composition. Suitable thermo-gelling compositions may include, for example, homopolymers, such as poly(N-methyl-N-n-propylacrylamide), poly(N-n-propylacrylamide), poly(N-methyl-N-isopropylacrylamide), poly(N-n-propylmethacrylamide), poly(N-isopropylacrylamide), poly(N,n-diethylacrylamide); poly(N-isopropylmethacrylamide), poly(N-cyclopropylacrylamide), poly(N-ethylmethyacrylamide), poly(N-methyl-N-ethylacrylamide), poly(N-cyclopropylmethacrylamide), and poly(N-ethylacrylamide). Still other examples of suitable thermogelling polymers may include cellulose ether derivatives, such as hydroxypropyl cellulose, methyl cellulose, hydroxypropylmethyl cellulose, and ethylhydroxyethyl cellulose. Moreover thermogelling polymers may be made by preparing copolymers between (among) monomers, or by combining such homopolymers with other water-soluble polymers, such as acrylic monomers (e.g., acrylic or methacrylic acid, acrylate or methacrylate, acrylamide or methacrylamide, and derivatives thereof).

In one particular embodiment of the present disclosure, for example, the composition is configured to rapidly form a gel when applied to the vagina. A “gel” is a colloid in which a disperse phase combines with a dispersion medium to produce a jelly-like, solid or semi-solid material. The gel may form in less than about one hour, in some embodiments less than about one minute, and in some embodiments, less than about 30 seconds. Among other things, such rapid gelation reduces the likelihood of leakage during use. In addition, because the gel may form intravaginally, it is more likely to retain its structure and shape over an extended period of time. In this manner, the gel may provide the prolonged release of a therapeutic agent that inhibits and/or treats vaginal infection. For instance, the gel may remain within the vagina for about 2 to about 48 hours to provide the desired effect to modulate the bladder microbiome of the subject.

Although a variety of compounds may be employed, water is usually employed as the dispersion medium for the gel to optimize biocompatibility. Other possible dispersion mediums include non-aqueous solvents, including glycols, such as propylene glycol, butylene glycol, triethylene glycol, hexylene glycol, polyethylene glycols, ethoxydiglycol, and dipropyleneglycol; alcohols, such as ethanol, n-propanol, and isopropanol; triglycerides; ethyl acetate; acetone; triacetin; and combinations thereof. Typically, the dispersion medium (e.g., water) constitutes greater than about 75 wt/vol %, in some embodiments greater than about 90 wt/vol %, and in some embodiments, from about 95 to about 99 wt/vol % of the composition.

The compositions of the present disclosure may also include an ion responsive compound. Such compounds are generally well known in the art, and tend to form a gel in the presence of certain ions or at a certain pH. For instance, one suitable class of ion responsive compounds that may be employed in the present disclosure is anionic polysaccharides. Anionic polysaccharides may form a three-dimensional polymer network that functions as the disperse phase of the gel. Generally speaking, anionic polysaccharides include polysaccharides having an overall anionic charge, as well as neutral polysaccharides that contain anionic functional groups.

Any of a variety of anionic polysaccharides capable of forming a gel when contacted with vaginal mucosa may be used in the present disclosure. Such gel-forming anionic polysaccharides are typically stable over the normal acidic pH values found in the vagina (e.g., from about 2.5 to about 5.5). For instance, some suitable examples of gel-forming anionic polysaccharides include natural gums, such as gellan gum and alginate gums (e.g., ammonium and alkali metal of salts of alginic acid); chitosan; carboxymethylcellulose, pectins, carrageenan, xantham gum, and derivatives or salts thereof. The particular type of anionic polysaccharide selected will depend, in part, on the nature of the composition and the other components used therein. For example, carrageenan is sensitive to particular types of cations, e.g., it typically gels in the presence of potassium but not sodium. Glycuronans, likewise, typically gel in the presence of divalent cations (e.g., Ca2+), but not monovalent cations (e.g., Na+). Xanthan gum may gel in the presence of divalent cations, but only at a relatively high pH.

Although any of the above-described anionic polysaccharides may be used in the present disclosure, gellan gum is particularly desired for use in the present disclosure, either alone or in combination with other gelling agents, because it is able to form a gel in the presence of a wide variety of different cations, including both monovalent and divalent cations. Gellan gum is intended to encompass any form of gellan, including native gellan, clarified gellan, deacylated gellan, nonacylated gellan (e.g., produced from genetically engineered bacteria), clarified gellan (the polysaccharide is fully or partially removed from the bacterial debris), chemically modified gellan, etc. Various types of gellan gums and methods for forming such gums are described in U.S. Pat. Nos. 4,326,052, 4,326,053, 4,377,636, 4,385,123, and 4,563,366. Suitable gellan gums are commercially available from a variety of different sources. For example, GELRITE™ gellan gum is available from Sigma-Aldrich Chemical Co. of St. Louis, MO, and is produced from a naturally occurring polysaccharide after deacylation and clarification. Deacylated gellan is also available from CP Kelco U.S., Inc. of Chicago, IL under the name KELCOGEL®.

Gellan gum may be either high or low acyl gellan. In the high acyl (or “native”) form, two acyl substituents, acetate and glycerate, are present. Both substituents are located on the same glucose residue and, on average, there is one glycerate per repeat unit and one acetate per every two repeat units. In the low acyl form, the acyl groups may be wholly or partially removed through deacylation. The degree of deacylation of deacylated gellan gums may be at least about 20%, in some embodiments at least about 50%, and in some embodiments, at least about 75%. Alternatively, the low acyl gellan gum may simply be “nonacylated” in that it is formed without acyl groups by genetically engineered bacteria. Regardless of the manner in which they are formed, low acyl gellan gums generally have a gelation temperature within the range 30 to 50° C., which may be particularly well suited for use in the present disclosure so that it may gel at body temperatures of about 37° C., but remain stable at typical storage and transportation temperatures of about 25° C. In addition, low acyl gellan gums are also firm and elastic, and thus may retain their shape after delivery to the vaginal cavity.

In most embodiments the gelling agent(s) are present in an amount of from about 0.01 to about 10.0 wt/vol %, in some embodiments from about 0.05 to about 5.0 wt/vol %, and in some embodiments, from about 0.1 to about 1.0 wt/vol % of the composition.

If desired, a gelling composition may be provided in any desired form (e.g., liquid, powder, etc.). In fact, one particular benefit of the composition is that it may be administered as a liquid, which allows for the selection of a wider variety of administration techniques than would otherwise be available for a solid or semi-solid gel. One technique that may be employed includes dispensing the composition through a liquid applicator, such as a syringe or tube, into the vaginal cavity. The administered volume of the composition may constitute a single dose or two or more doses. Although not necessarily required, the composition of may also be sterilized prior to administration. Sterilization may be accomplished by any technique known in the art, such as using a gas (e.g., ethylene oxide), radiation (e.g., gamma), or heat (autoclaving). If desired, the composition may be subjected to one or more filtration steps prior to sterilization to help remove contaminants.

Antimicrobial Agents

In some embodiments, the composition may include one or more antimicrobial agents to increase shelf life. Some suitable antimicrobial agents that may be used in the present disclosure include traditional antimicrobial agents. As used herein, “traditional antimicrobial agents” means compounds that have been historically recognized by regulatory bodies as providing an antimicrobial effect, such as those listed in the European Union's Annex V list of preservatives allowed in cosmetics products. Traditional antimicrobial agents include, but are not limited to: propionic acid and salts thereof; salicylic acid and salts thereof; sorbic acid and salts thereof; benzoic acid and salts and esters thereof; formaldehyde; paraformaldehyde; o-phenylphenol and salts thereof; zinc pyrithione; inorganic sulfites; hydrogen sulfites; chlorobutanol; benzoic parabens, such as methylparaben, propylparaben, butylparaben, ethylparaben, isopropylparaben, isobutylparaben, benzylparaben, sodium methylparaben and sodium propylparaben; dehydroacetic acid and salts thereof; formic acid and salts thereof; dibromohexamidine isethionate; thimerosal; phenylmercuric salts; undecylenic acid and salts thereof; hexetidine; 5-bromo-5-nitro-1,3-dioxane; 2-bromo-2-nitropropane-1,3,-diol; dichlorobenzyl alcohol; triclocarban; p-chloro-m-cresol; triclosan; chloroxylenol; imidazolidinyl urea; polyaminopropyl biguanide; phenoxyethanol, methenamine; quaternium-15; climbazole; DMDM hydantoin; benzyl alcohol; piroctone olamine; bromochlorophene; o-cymen-5-ol; methylchloroisothiazolinone; methylisothiazolinone; chlorophene; chloroacetamide; chlorhexidine; chlorhexidine diacetate; chlorhexidine digluconate; chlorhexidine dihydrochloride; phenoxyisopropanol; alkyl (C₁₂-C₂₂) trimethyl ammonium bromide and chlorides; dimethyl oxazolidine; diazolidinyl urea; hexamidine; hexamidine diisethionate; glutaral; 7-ethylbicyclooxazolidine; chlorphenesin; sodium hydroxymethylglycinate; silver chloride; benzethonium chloride; benzalkonium chloride; benzalkonium bromide; benzylhemiformal; iodopropynyl butylcarbamate; ethyl lauroyl arginate HCl; citric acid and silver citrate.

Other antimicrobial agents that may be added to the compositions of the present disclosure include non-traditional antimicrobial agents that are known to exhibit antimicrobial effects in addition to their primary functions, but that have not historically been recognized as antimicrobial agents by regulatory bodies (such as on the European Union's Annex V list). Examples of these non-traditional antimicrobial agents include, but are not limited to, hydroxyacetophenone, caprylyl glycol, sodium coco-PG dimonium chloride phosphate, phenylpropanol, lactic acid and salts thereof, caprylhydroxamic acid, levulinic acid and salts thereof, sodium lauroyl lactylate, phenethyl alcohol, sorbitan caprylate, glyceryl caprate, glyceryl caprylate, ethylhexylglycerin, p-anisic acid and salts thereof, gluconolactone, decylene glycol, 1,2-hexanediol, glucose oxidase and lactoperoxidase, leuconostoc/radish root ferment filtrate and glyceryl laurate.

The amount of the antimicrobial agents in the compositions is dependent on the relative amounts of other components present within the composition. For example, in some embodiments, an antimicrobial agent can be present in the compositions in an amount between about 0.001% to about 5% (by total weight of the composition), in some embodiments between about 0.01 to about 3% (by total weight of the composition), and in some embodiments, between about 0.05% to about 1.0% (by total weight of the composition). In some embodiments, the antimicrobial agent can be present in the composition in an amount less than 0.2% (by total weight of the composition). However, in some embodiments, the composition can be substantially free of any antimicrobial agents. Thus, in some embodiments, the composition does not include a traditional antimicrobial agent or a non-traditional antimicrobial agent.

Other suitable additives that may be included in the compositions of the present disclosure include compatible colorants, deodorants, emulsifiers, anti-foaming agents (when foam is not desired), lubricants, skin conditioning agents, skin protectants and skin benefit agents (e.g., aloe vera and tocopheryl acetate), solvents (e.g., water soluble glycol and glycol ethers, glycerin, water soluble polyethylene glycols, water soluble polyethylene glycol ethers, water soluble polypropylene glycols, water soluble polypropylene glycol ethers, dimethylisosorbide), solubilizing agents, suspending agents, builders, (e.g., alkali and alkaline earth metal salts of carbonate, bicarbonate, phosphate, hydrogen phosphate, dihydrogen phosphate, sulfate hydrogen sulfate), wetting agents, pH adjusting ingredients (a suitable pH range of the compositions can be from about 3.5 to about 8), chelators, propellants, dyes and/or pigments, and combinations thereof.

The compositions of the present disclosure may be applied to a suitable substrate, which in-turn may be used to apply the therapeutic agent to a user. Suitable applicators include a web, such as a wet laid tissue web or air laid web, gauze, cotton swab, transdermal patch, container or holder. Particularly preferred applicators include fibrous webs, including flushable and non-flushable cellulosic webs and nonwoven webs of synthetic fibrous material. Useful webs may be wet laid, air laid, meltblown, or spunbonded. Suitable synthetic fibrous material includes meltblown polyethylene, polypropylene, copolymers of polyethylene and polypropylene, bicomponent fibers including polyethylene or polypropylene, and the like. Useful nonwoven webs may be meltblown, coform, spunbond, airlaid, hydroentangled nonwovens, spunlace, bonded carded webs.

In certain embodiments, particularly those in which the composition is applied to a web, it may be desirable that the formulation provide certain physical attributes, such as having a smooth, lubricious, non-greasy feel; the ability to at least partially transfer from the web to the user; the capability to be retained on the web at about room temperature; or the ability to be compatible with the web manufacturing process. In certain embodiments, it is preferred that at least a portion of the composition is transferred from the tissue to the user in use.

The composition may be applied to a web during formation of the web or after the web has been formed and dried, often referred to as off-line or post-treatment. Suitable methods of applying the composition to a web include methods known in the art such as gravure printing, flexographic printing, spraying, WEKO™, slot die coating, or electrostatic spraying. One particularly preferred method of off-line application is rotogravure printing.

### Test Methods

### Method for High Throughput Assay Screening of Prebiotics

1. Create a 2% w/v stock of prebiotic or positive control (glucose/dextrose) to be tested in API 50 CHL media (Biomerieux, Marcy-I'Étoile, France). API 50 CHL media contains minimal amounts of carbon for growth, and a bromocresol purple as a pH indicator of fermentation. Filter sterilize media with a 0.2 μM filter. Store at 4-6° C. until use.
2. Add 180 μL of media containing the prebiotic to a sterile, flat bottom 96-well microtiter plate (Corning, Corning, NY).
3. From a freezer stock subculture the bacteria twice into De Man, Rogosa and Sharpe (MRS) broth (BD Difco, Becton Dickinson, Franklin Lakes, NJ), 37° C., stationary, anaerobic, overnight.
4. From the last subculture, plate onto an MRS agar plate (Teknova, Hollister, CA) and grow 37° C., stationary, anaerobic, overnight.
5. Create a bacterial suspension by using a sterile tipped swab to transfer colonies of bacteria into a PBS suspension blank to a 0.5 MacFarland (S. anginosus or E. faecalis) or a 1 MacFarland (Lactobacillus species) from the MRS agar plate.
6. Enumerate the starting culture.
7. Inoculate the media alone and media containing the prebiotic or positive control with 20 μL of the bacterial suspension.
8. Place the plate into a spectrophotometer (Molecular Devices, San Jose, CA) and read the plate every 20 minutes for 24-48 hours. Read wavelengths (OD 430, OD 590, and OD 660). A color change to yellow caused by a drop in pH will be read by an increase in the OD430 value and a decrease in the OD590 value and is indicative of bacterial fermentation. An increase in OD660 values is indicative of an increase in turbidity and bacterial growth.

### Method for Plate Assay Screening of Prebiotics

1. An in house bacterial agar was created based on MRS agar (Kaplan and Hutkins, 2000) where the glucose is substituted for the prebiotic to be tested (either dextran MW ˜6,000, alpha-cyclodextrin, glucomannan, or pectin), bromocresol purple was included as a pH indicator.
2. Bacteria were enumerated on each agar and color changes were recorded.
   Method for Competition Assay (Modified from “In Vitro Evaluation of Nutrients that Selectively Confer a Competitive Advantage to Lactobacilli” Vongsa et al, Beneficial Microbes 2016)
1. Create 2% w/v stock of prebiotic or positive control (glucose/dextrose) as described above.
2. From a freezer stock subculture the bacteria twice into De Man, Rogosa and Sharpe (MRS) broth (BD Difco, Becton Dickinson, Franklin Lakes, NJ), 37° C., stationary, anaerobic, overnight.
3. From the last subculture, plate onto an MRS agar plate for L. crispatus KC18-1173-1 or blood agar for S. anginosus KC18-1131-3B, grow 37° C., stationary, anaerobic, overnight.
4. Create a bacterial suspension by using a sterile tipped swab to transfer colonies of bacteria into a PBS suspension blank to a 2 MacFarland for both species.
5. Enumerate the starting culture.
6. Add 9.8 mL of prebiotic media into a 15 mL conical tube.
7. Add 100 μL of the diluted bacteria to the media, incubate at 37° C., anaerobic, 48 hours.
8. Harvest 100 μL from the tube containing the prebiotic and plate onto MRS agar and tryptic soy agar (TSA).
9. Enumerate the bacteria on the MRS agar (both the L. crispatus and the S. anginosus). Small colonies are counted as S. anginosus and large colonies are counted as L. crispatus.
10. Enumerate the bacteria on the TSA plate. Lactobacillus does not grow well on TSA, whereas S. anginosus does.
11. Growth of the bacteria in the negative control (CHL media without prebiotic) was subtracted from the growth in the presence of the prebiotic or formulation.

### Therapeutic Effect Protocol

Colonies of L. crispatus and S. anginosus were tested against in the competition method assay test method as described above. The therapeutic effect was calculated by comparing the colony forming units recovered after the competition assay of both L. crispatus and S. anginosus and calculating the ratio of L. crispatus/S. anginosus. A larger number indicates an increase in L. crispatus and/or a decrease in S. anginosus and a smaller number indicates a decrease in L. crispatus and/or an increase in S. anginosus.

### Embodiments

In view of the foregoing description and examples, the present disclosure provides the following embodiments.

Embodiment 1: A method for modulating a bladder microbiome in a subject to improve bladder health, the method comprising: providing a composition, the composition comprising: a carrier; and a bladder therapeutic agent, the bladder therapeutic agent comprising isomaltulose; administering the composition to a urogenital region of the subject; and promoting a growth of Lactobacillus crispatus relative to Streptococcus anginosus within the bladder microbiome to modulate the bladder microbiome to improve bladder health.

Embodiment 2: The method of embodiment 1, wherein promoting a growth of Lactobacillus crispatus to Streptococcus anginosus provides a therapeutic effect of at least 100.

Embodiment 3: The method of embodiment 1 or 2, wherein administering the composition to the urogenital region of the subject comprises administering the composition to the urethra or periurethral area of the subject.

Embodiment 4: The method of any one of the preceding embodiments, wherein the bladder therapeutic agent comprises from about 0.01% to about 10.0 wt/vol % of the composition.

Embodiment 5: The method of any one of the preceding embodiments, wherein the carrier comprises greater than about 90.0 wt/vol % of the composition.

Embodiment 6: The method of any one of the preceding embodiments, wherein the carrier comprises water.

Embodiment 7: The method of any one of the preceding embodiments, further comprising: applying the composition to a substrate.

Embodiment 8: The method of embodiment 7, wherein the substrate comprises a wipe or at least a portion of an absorbent article.

Embodiment 9: The method of any one of the preceding embodiments, wherein the composition further comprises at least one of a surfactant, an ester, a humectant, a pH adjuster, a rheology modifier, a gelling agent, and an antimicrobial agent.

Embodiment 10: The method of any one of the preceding embodiments, wherein the composition is in the form of a liquid, gel, cream, or spray.

Embodiment 11: A method for preventing or treating overactive bladder, urinary urge incontinence, or urinary tract infections in a subject, the method comprising: providing a composition, the composition comprising: a carrier; and a bladder therapeutic agent, the bladder therapeutic agent comprising isomaltulose; and administering the composition to a urogenital region of the subject to prevent or treat overactive bladder, urinary urge incontinence, or urinary tract infections in the subject.

Embodiment 12: The method of embodiment 11, wherein administering the composition to the urogenital region of the subject modulates a bladder microbiome by promoting a growth of Lactobacillus crispatus relative to Streptococcus anginosus within the bladder microbiome.

Embodiment 13: The method of embodiment 12, wherein promoting a growth of Lactobacillus crispatus to Streptococcus anginosus provides a therapeutic effect of at least 100.

Embodiment 14: The method of any one of embodiments 11-13, wherein administering the composition to the urogenital region of the subject comprises administering the composition to the urethra or periurethral area of the subject.

Embodiment 15: The method of any one of embodiments 11-14, wherein the bladder therapeutic agent comprises from about 0.01% to about 10.0 wt/vol % of the composition.

Embodiment 16: The method of any one of embodiments 11-15, wherein the carrier comprises greater than about 90.0 wt/vol % of the composition.

Embodiment 17: The method of any one of embodiments 11-16, wherein the carrier comprises water.

Embodiment 18: The method of claim 11, further comprising: applying the composition to a substrate.

Embodiment 19: The method of embodiment 18, wherein the substrate comprises a wipe or at least a portion of an absorbent article.

Embodiment 20: The method of any one of embodiments 11-19, wherein the composition is in the form of a liquid, gel, cream, or spray.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for modulating a bladder microbiome in a subject to improve bladder health, the method comprising:
providing a composition, the composition comprising:
a carrier; and
a bladder therapeutic agent, the bladder therapeutic agent comprising isomaltulose;
administering the composition to a urogenital region of the subject; and
promoting a growth of Lactobacillus crispatus relative to Streptococcus anginosus within the bladder microbiome to modulate the bladder microbiome to improve bladder health.

2. The method of claim 1, wherein promoting a growth of Lactobacillus crispatus to Streptococcus anginosus provides a therapeutic effect of at least 100.

3. The method of claim 1, wherein administering the composition to the urogenital region of the subject comprises administering the composition to the urethra or periurethral area of the subject.

4. The method of claim 1, wherein the bladder therapeutic agent comprises from about 0.01% to about 10.0 wt/vol % of the composition.

5. The method of claim 1, wherein the carrier comprises greater than about 90.0 wt/vol % of the composition.

6. The method of claim 1, wherein the carrier comprises water.

7. The method of claim 1, further comprising:
applying the composition to a substrate.

8. The method of claim 7, wherein the substrate comprises a wipe or at least a portion of an absorbent article.

9. The method of claim 1, wherein the composition further comprises at least one of a surfactant, an ester, a humectant, a pH adjuster, a rheology modifier, a gelling agent, and an antimicrobial agent.

10. The method of claim 1, wherein the composition is in the form of a liquid, gel, cream, or spray.

11. A method for preventing or treating overactive bladder, urinary urge incontinence, or urinary tract infections in a subject, the method comprising:
providing a composition, the composition comprising:
a carrier; and
a bladder therapeutic agent, the bladder therapeutic agent comprising isomaltulose; and
administering the composition to a urogenital region of the subject to prevent or treat overactive bladder, urinary urge incontinence, or urinary tract infections in the subject.

12. The method of claim 11, wherein administering the composition to the urogenital region of the subject modulates a bladder microbiome by promoting a growth of Lactobacillus crispatus relative to Streptococcus anginosus within the bladder microbiome.

13. The method of claim 12, wherein promoting a growth of Lactobacillus crispatus to Streptococcus anginosus provides a therapeutic effect of at least 100.

14. The method of claim 11, wherein administering the composition to the urogenital region of the subject comprises administering the composition to the urethra or periurethral area of the subject.

15. The method of claim 11, wherein the bladder therapeutic agent comprises from about 0.01% to about 10.0 wt/vol % of the composition.

16. The method of claim 11, wherein the carrier comprises greater than about 90.0 wt/vol % of the composition.

17. The method of claim 11, wherein the carrier comprises water.

18. The method of claim 11, further comprising:
applying the composition to a substrate.

19. The method of claim 18, wherein the substrate comprises a wipe or at least a portion of an absorbent article.

20. The method of claim 11, wherein the composition is in the form of a liquid, gel, cream, or spray.
